(19) United States
(12) Patent Application Publication
(10) Pub. No.: US 2023/0131065 A1
(43) Pub. Date: Apr. 27, 2023

(54) PROCESS FOR FUSION OF ESSENTIAL TRACE ELEMENTS INTO STANDARDIZED HERBAL EXTRACTS

(71) Applicant: Sanjay TAMOLI, Maharashtra (IN)

(72) Inventor: Sanjay TAMOLI, Maharashtra (IN)

(21) Appl. No.: 17/905,627

(22) PCT Filed: Mar. 1, 2021
(86) PCT No.: PCT/IB2021/051672

§ 371 Date: Sep. 2, 2022

(30) Foreign Application Priority Data
Mar. 2, 2020 (IN) 202021008818

(51) Int. Cl. A61K 36/9066

(52) U.S. Cl. CPC A61K 36/9066; A61K 2236/15

(57) ABSTRACT

The present invention relates to a process for standardized herbal extracts. More specifically, the invention provides a process for fusion of essential trace elements and the phyto-constituents in the standardized herbal extracts. The process comprises the sequential steps of: a) selecting an appropriate essential trace element to be fused in the standardized herbal extract; b) micronizing the standardized herbal extracts; and c) bio-ligation by forming natural bonds (bioligands) in the standardized herbal extract between the active phyto-constituent and the essential trace element.

### PRIORITY

This application claims the benefit of Indian complete application number 202021008818 dated 2 Mar. 2020 entitled, ‘A process for fusion of essential trace elements into standardized herbal extracts’, the contents of which are incorporated herein by reference.

### TECHNICAL FIELD OF THE INVENTION

The present invention relates to a process for standardized herbal extracts. More specifically, the invention provides a sequential step by step process of Fusion, Micronization and Bioligation (FMB process) of essential trace elements and the phyto-constituents in standardized herbal extracts.

### BACKGROUND OF THE INVENTION

Use of herbal extracts has been ever growing over the past few decades. The major source of information on the use of plant materials for prevention and treatment of various ailments in humans and animals is the traditional knowledge based on literature, experience and experiments. The efficacy and safety of these herbs has been always attributed to using the plant material as a whole. The reason for this holistic approach was with an understanding of providing synergy in various plant constituents to provide the highest degree of efficacy desired from the plant.

Over the past few decades plant based extracts (phyto-extracts) have been extensively used globally. Extraction using various methods and mediums (solvents to say) are done with the primary intention to provide higher concentration of the possible active plant constituents (at times only marker compounds) in a consistent manner. These extracts are referred to as standardized herbal extracts (SHE) as they are standardized to contain specific plant constituent (volatile oil, alkaloids, polyphenols, tannins etc.). While standardization ensures consistent quality standards avoiding natural variables like geography, climate etc. it does not essentially reflect and ensure efficacy.

The whole process of extraction with different methods and medias (solvents) have a very high possibility of removing or eliminating many essential fractions/constituents which have a definite role in determining the effect and ensuring safety of the extract. Extraction process may concentrate a few constituents at the expense of other important ones. This changes the complete natural balance of the constituents in the plant (herbal material). One of the major loss/lacuna in these extraction processes is the essential trace elements present in the basic plant material.

Herbs/Plants contain a variety of essential trace elements (ETE) in measurable concentration. These ETE are present in most bioavailable form and have a very vital role in the overall pharmacological action of the plant. While most of the scientific studies on medicinal plants primarily focus on their organic contents viz. essential oils, glycosides, alkaloids, poly phenols etc. the role of trace elements is usually undermined. However apart from these organic compounds it is now well established that many trace elements play a vital role in the pharmacological/therapeutic action of the herb. These trace elements are present at varying concentrations in different plant parts (root, stem, flowers etc.) Their concentration shows variations depending preferential absorbability for a specific trace element (natural plant preference to carry out its metabolic activity). Also geography (soil quality and climate condition) plays a role just as the organic constituents.

The essential trace elements not only have the role or being merely essential nutritional components but also have pivotal role in carrying out anti-inflammatory reactions, micro-enzymatic reactions, anti-oxidant reactions amongst others. This is applicable more to plants that have therapeutic potential rather that nutritional potential. They also have important role in enhancing the absorption and improving the bioavailability of the phyto-constituents. Scientific studies prove the affinity of trace elements to particular tissue/cell types as well.

Therefore, as we can deduce from these benefits of preserving ETE in the standardized herbal extracts (SHE) it appears relevant to develop methods to do so. Accordingly, there is a need for developing processes or changing methods in the current extraction techniques to preserve the ETE.

### SUMMARY OF THE INVENTION

The present invention provides a process for fusion of essential trace elements and the phyto-constituents in a standardized herbal extracts.

In an embodiment of the present invention, standardized herbal extract comprising of a plant constituent fused with an essential trace element is provided with high bioavailability and better pharmacological efficacy.

In another embodiment of the present invention, process for obtaining a standardized herbal extract containing a plant constituent fused with an essential trace element using the sequential technique of fusion, micronization and bioligation is provided.

### DETAILED DESCRIPTION OF THE INVENTION

The present invention relates to a process for standardized herbal extracts preparation.

The terms used in the specification are defined as follows.

The use of the terms “a” and “an” and “the” and similar referents in the context of describing the elements (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential to the practice of the invention.

As used herein, “about” will be understood by persons of ordinary skill in the art and will vary to some extent depending upon the context in which it is used. If there are uses of the term in which are not clear to persons of ordinary skill in the art, given the context in which it is used, “about” will mean up to plus or minus 10% of the particular term.

As used in the present invention, the term “standardized herbal extract” is defined as a herbal extract that has one or more component present in a specific, guaranteed amount.

A trace element is a chemical element present in minute quantities; especially: a micronutrient (such as iron, calcium, and zinc) with an optimum daily intake of typically less than 100 milligrams a day. A trace element is considered “essential” if a dietary deficiency of the element consistently results in a suboptimal biologic function in a host, comprising plant, animal and/or human that is preventable or reversible by intake of physiologic amounts of that element. The following elements are needed by a living organism only in trace or ultra-trace amounts and are also termed “essential trace elements” in accordance with the present invention: arsenic (As), boron (B), chromium (Cr), cobalt (Co), copper (Cu), iron (Fe), fluorine (F), lithium (Li), iodine (I), manganese (Mn), molybdenum (Mo), nickel (Ni), selenium (Se), strontium (Sr), silicon (Si), sulphur (S), tin (Sn), vanadium (V), zinc (Zn) and optionally the rare earth elements.

The term “micronization” as used herein refers to the process of reducing the average diameter of a solid material's particles. Usually, the term micronization is used when the particles that are produced from only a few micrometers (typically less than 100 μm) to the nanometer range in diameter.

Extraction, as the term used, involves the separation of medicinally active portions of plant or animal tissues from the inactive or inert components by using selective solvents in standard extraction procedures. The products so obtained from plants are relatively impure liquids, semisolids or powders intended only for oral or external use. These include classes of preparations known as decoctions, infusions, fluid extracts, tinctures, pilular (semisolid) extracts and powdered extracts.

The purposes of standardized extraction procedures are to attain the therapeutically desired portion and to eliminate the inert material by treatment with a selective solvent. The extract thus obtained may be ready for use as a medicinal agent in the form of tinctures and fluid extracts, or dry powder extracts it may be further processed to be incorporated in any dosage form such as tablets or capsules or liquids etc., or it may be fractionated to isolate individual chemical entities. Thus, standardization of extraction procedures contributes significantly to the final quality of the extract.

Various extraction processes include maceration, infusion, digestion, decoction, percolation, hot continuous extraction (Soxhlet), aqueous Alcoholic Extraction by Fermentation, counter-current extraction, ultrasound extraction (Sonication), supercritical fluid extraction and the like. Selection of extraction process is important and based on the kind of extract required, the extraction process is selected.

In maceration, the whole or coarsely powder is placed in a stoppered container with the solvent and allowed to stand at room temperature for a period of at least 3 days with frequent agitation until the soluble matter has dissolved. The mixture then is strained, the marc (the damp solid material) is pressed, and the combined liquids are clarified by filtration or decantation after standing.

Fresh infusions are prepared by macerating the crude for a short period of time with cold or boiling water. These are dilute solutions of the readily soluble constituents of crude material.

Digestion is a form of maceration in which gentle heat is used during the process of extraction. It is used when moderately elevated temperature is not objectionable. The solvent efficiency of the menstruum is thereby increased.

In decoction, the crude is boiled in a specified volume of water for a defined time; it is then cooled and strained or filtered. This procedure is suitable for extracting water-soluble, heat-stable constituents. This process is typically used in preparation of Ayurvedic extracts called “quath” or “kawath”. The starting ratio of crude to water is fixed, e.g. 1:4 or 1:16; the volume is then brought down to one-fourth its original volume by boiling during the extraction procedure. Then, the concentrated extract is filtered and used as such or processed further.

In hot continuous extraction, the finely ground crude is placed in a porous bag or “thimble” made of strong filter paper, which is placed in chamber of the Soxhlet apparatus. The extracting solvent in flask is heated, and its vapors condense in condenser. The condensed extractant drips into the thimble containing the crude, and extracts it by contact. When the level of liquid in chamber rises to the top of siphon tube, the liquid contents of chamber siphon into flask. This process is continuous and is carried out until a drop of solvent from the siphon tube does not leave residue when evaporated. The advantage of this method, compared to previously described methods, is that large amounts of crude can be extracted with a much smaller quantity of solvent. This effect tremendous economy in terms of time, energy and consequently financial inputs. At small scale, it is employed as a batch process only, but it becomes much more economical and viable when converted into a continuous extraction procedure on medium or large scale.

Aqueous Alcoholic Extraction by Fermentation involves soaking the crude, in the form of either a powder or a decoction, for a specified period of time, during which it undergoes fermentation and generates alcohol in situ; this facilitates the extraction of the active constituents contained in the plant material. The alcohol thus generated also serves as a preservative. If the fermentation is to be carried out in an earthen vessel, it should not be new: water should first be boiled in the vessel. In large-scale manufacture, wooden vats, porcelain jars or metal vessels are used in place of earthen vessels.

In counter-current extraction (CCE), wet raw material is pulverized using toothed disc disintegrators to produce fine slurry. In this process, the material to be extracted is moved in one direction (generally in the form of a fine slurry) within a cylindrical extractor where it comes in contact with extraction solvent. The further the starting material moves, the more concentrated the extract becomes. Complete extraction is thus possible when the quantities of solvent and material and their flow rates are optimized.

The ultrasound extraction involves the use of ultrasound with frequencies ranging from 20 kHz to 2000 kHz; this increases the permeability of cell walls and produces cavitation. Although the process is useful in some cases, like extraction of rauwolfia root, its large-scale application is limited due to the higher costs. One disadvantage of the procedure is the occasional but known deleterious effect of ultrasound energy (more than 20 kHz) on the active constituents of medicinal plants through formation of free radicals and consequently undesirable changes in the molecules.

Supercritical fluid extraction (SFE) is an alternative sample preparation method with general goals of reduced use of organic solvents and increased sample throughput. The factors to consider include temperature, pressure, sample volume, analyte collection, modifier (cosolvent) addition, flow and pressure control, and restrictors. Generally, cylindrical extraction vessels are used for SFE and their performance is good beyond any doubt.

The present invention will now be described with reference to the accompanying embodiments of the invention. This invention may, however, be embodied in many different forms and should not be construed as being limited to the embodiment set forth herein. Rather, the embodiment is provided so that this disclosure will be thorough, and will fully convey the scope of the invention to those skilled in the art.

In an embodiment of the present invention, standardized herbal extract comprising of a plant constituent fused with an essential trace element is provided with high bioavailability and better pharmacological efficacy of the plant constituent.

In another embodiment of the present invention, a three step process, known as FMB process is provided for obtaining a standardized herbal extract containing a plant constituent fused with an essential trace element, said process comprising the sequential steps of:
a) Fusion: selecting an appropriate essential trace element to be fused in the standardized herbal extract;
b) Micronization: micronizing the standardized herbal extracts; and
c) Bio-ligation: by forming natural bonds (bioligands) in the standardized herbal extract between phyto-constituent and the essential trace element.

Fusion ensures replenishment of extract with their respective cofactor (ETE) those are lost during conventional extraction of botanicals. The process provides greater surface area of the extract matrix, this enhances the absorption and provides greater efficacy of the respective extract used for the intended application. It may help in reducing the dosage of the extract for its targeted activity with higher efficacy and therefore contribute to reduce the cost of the treatment. The process of “bioligation” facilitates selective absorption of the active moiety of herbal extract at the target site resulting in effective absorption and hence better efficacy to achieve desired activity. It also facilitates stable complexes of the ETE with the phyto-constituents. Same herbal extract can be utilized for different therapeutic action based on the phyto-mineral bioligand complex developed. Process ensures greater stability of the FMB Extract.

The primary action of many plant extracts is through the anti-oxidant pathway. FMB extracts provide higher anti-oxidant potentials as compared to SHE. The quantity of ETE in these extracts is in the range what was essentially present in the original plant material from which extraction is done. FMB extracts can be used in all dosage routs just as SHE.

The current invention does not intend to merely fortify extracts with trace elements. It also does not in any way provide trace elements in the concentration to suffice the daily nutritional requirements. While ETE are essential for the desired phyto activity of the medicinal plants, toxic element especially heavy metals need to be avoided. FMB process ensures that the processed SHE does not have traces of toxic elements and the same are in the permissible limits as regulated by the highest quality standards for herbal extracts.

The foregoing examples are illustrative embodiments and are merely exemplary. A person skilled in the art may make variations and modifications without deviating from the spirit and scope of the invention. All such modifications and variations are intended to be included within the scope of the claims.

### Example: Fusion, Micronization and Bioligation Process According to the Invention

Curcuma longa Extract containing Curcuminoids as active phytoconstituents along with Zinc as an essential trace element in the form of fused micronized and bioligated complex helped in enhancing the Oxygen Radical Absorbance Capacity activity when compared with conventional Curcuma longa extract.

It will be understood that various modifications may be made to the aspects disclosed herein. Therefore, the above description should not be construed as limiting, but merely as exemplifications of preferred embodiments. Other arrangements and methods may be implemented by those skilled in the art without departing from the scope and spirit of this invention.

I claim:

1. A process for fusion of an essential trace element and phyto-constituents present in the standardized herbal extract, said process comprising the sequential steps of:
a) selecting an appropriate essential trace element to be fused in the standardized herbal extract;
b) micronizing the standardized herbal extracts; and
c) bio-ligation by forming natural bonds (bioligands) in the standardized herbal extract between the phyto-constituents and the essential trace element.

2. The process as claimed in claim 1 wherein, the micronizing step is performed by but not limited to continuous and incremental trituration milling and colloidal techniques.

3. A standardized herbal extract comprising of a plant constituent fused with an essential trace element, wherein, the plant constituent is fused, micronized and bioligated with said essential trace element.
